(19) United States
(12) Patent Application Publication
(10) Pub. No.: US 2024/0122292 A1
(43) Pub. Date: Apr. 18, 2024

(54) SHOE WITH ANKLE SUPPORT

(71) Applicant: REY TUASON, Toronto (CA)

(72) Inventor: REY TUASON, Toronto (CA)

(21) Appl. No.: 17/967,972

(22) Filed: Oct. 18, 2022

(51) Int. Cl. A43B 7/24; A43B 7/20; A61F 5/01

(52) U.S. Cl. CPC A43B 7/24; A43B 7/20; A61F 5/0111

(57) ABSTRACT

The shoe with ankle support is a therapeutic structure. The shoe with ankle support is adapted for use with an ankle of a foot of a patient. The shoe with ankle support is worn around the ankle of the foot of the patient. The shoe with ankle support comprises a plurality of inserts and a footwear structure. Each insert selected from the plurality of inserts is inserted into the footwear structure. The footwear structure is worn by the patient such that the footwear structure encloses both the foot and the ankle of the patient. Each insert selected from the plurality of inserts is sandwiched between the ankle and the interior of the footwear structure. Each insert selected from the plurality of inserts forms a brace that prevents the ankle from rolling in a lateral direction.

### CROSS REFERENCES TO RELATED APPLICATIONS

Not Applicable

### STATEMENT REGARDING FEDERALLY SPONSORED RESEARCH

Not Applicable

### REFERENCE TO APPENDIX

Not Applicable

### BACKGROUND OF THE INVENTION

### Field of the Invention

The present invention relates to the field of ankle-joint supports or holders. (A43B7/20)

### Background

While footwear comes in many styles and forms, most commonly available footwear shares certain common characteristics including, but not limited to, an upper and a sole. The upper and sole are generally said to be attached at the feather. If the feather is reinforced with an additional piece of material, the additional piece of material is referred to as a welt. The upper is divided into a quarter region and a vamp region. The superior edge of the upper is called the top line (or collar). A foot typically enters footwear through the top line. The quarter region covers the heel of the foot. The vamp region covers the toes and attaches to the quarter. The sole forms a pedestal between the foot and the ground. The sole is further defined with an insole and an outsole. The insole is the surface of the sole that is proximal to the foot when the footwear is worn normally. The outsole is the surface of the sole that is distal from the insole. The sole is often fitted with a heel which is a structure that is designed to raise the quarter portion of the footwear above the vamp. The tongue is a flap of material that is attached to the upper such that the tongue is positioned between a foot and the upper. The tongue is attached to the upper at the frenulum.

### SUMMARY OF INVENTION

The shoe with ankle support is a therapeutic structure. The shoe with ankle support is adapted for use with an ankle of a foot of a patient. The shoe with ankle support is worn around the ankle of the foot of the patient. The shoe with ankle support comprises a plurality of inserts and a footwear structure. Each insert selected from the plurality of inserts into the footwear structure. The footwear structure is worn by the patient such that the footwear structure encloses both the foot and the ankle of the patient. Each insert selected from the plurality of inserts is sandwiched between the ankle and the interior of the footwear structure. Each insert selected from the plurality of inserts forms a brace that prevents the ankle from rolling in a lateral direction.

These together with additional objects, features and advantages of the shoe with ankle support will be readily apparent to those of ordinary skill in the art upon reading the following detailed description of the presently preferred, but nonetheless illustrative, embodiments when taken in conjunction with the accompanying drawings.

In this respect, before explaining the current embodiments of the shoe with ankle support in detail, it is to be understood that the shoe with ankle support is not limited in its applications to the details of construction and arrangements of the components set forth in the following description or illustration. Those skilled in the art will appreciate that the concept of this disclosure may be readily utilized as a basis for the design of other structures, methods, and systems for carrying out the several purposes of the shoe with ankle support.

It is therefore important that the claims be regarded as including such equivalent construction insofar as they do not depart from the spirit and scope of the shoe with ankle support. It is also to be understood that the phraseology and terminology employed herein are for purposes of description and should not be regarded as limiting.

### BRIEF DESCRIPTION OF DRAWINGS

The accompanying drawings, which are included to provide a further understanding of the invention are incorporated in and constitute a part of this specification, illustrate an embodiment of the invention and together with the description serve to explain the principles of the invention. They are meant to be exemplary illustrations provided to enable persons skilled in the art to practice the disclosure and are not intended to limit the scope of the appended claims.

FIG. 1 is a perspective view of an embodiment of the disclosure.

FIG. 2 is a side view of an embodiment of the disclosure.

FIG. 3 is a rear view of an embodiment of the disclosure.

FIG. 4 is a top view of an embodiment of the disclosure.

FIG. 5 is an in-use view of an embodiment of the disclosure.

FIG. 6 is a detail view of an embodiment of the disclosure.

### DETAILED DESCRIPTION OF THE EMBODIMENT

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary or the following detailed description.

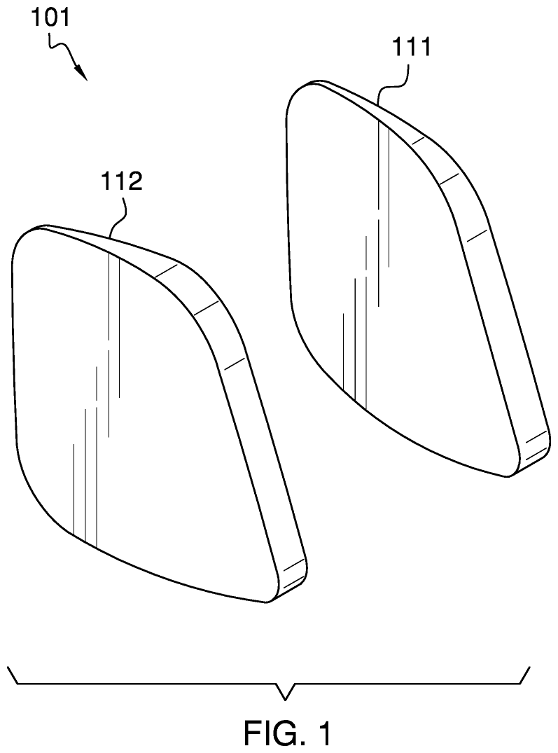

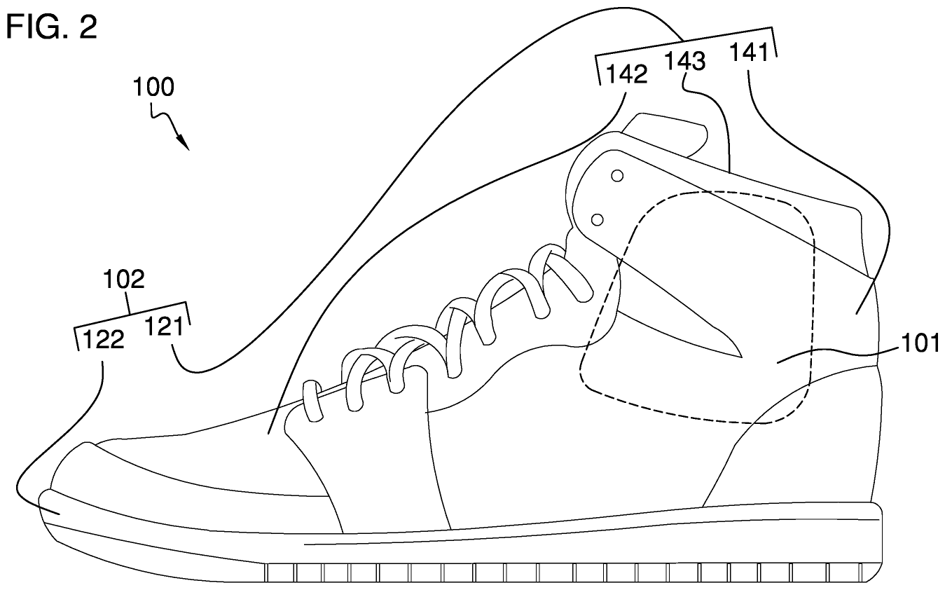

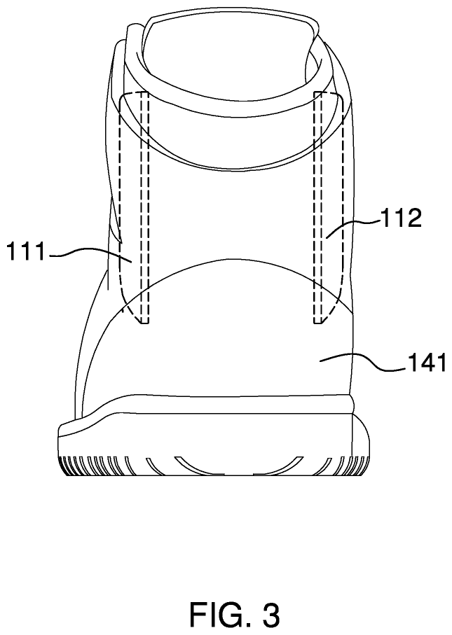

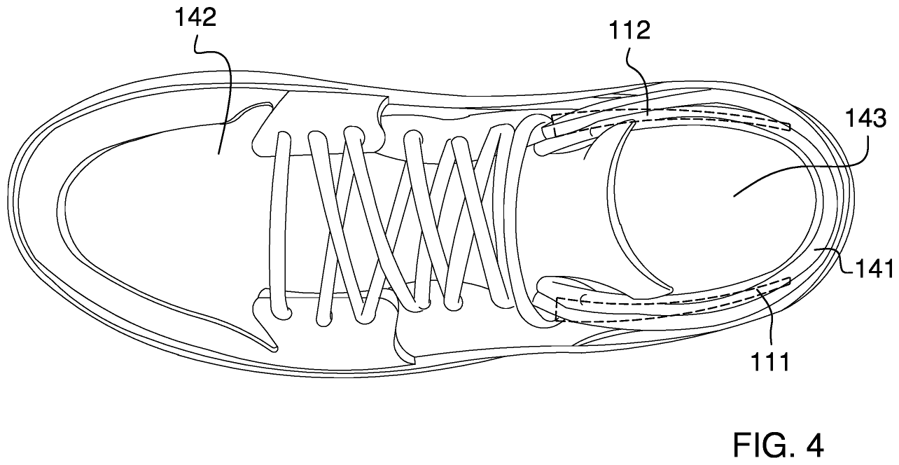

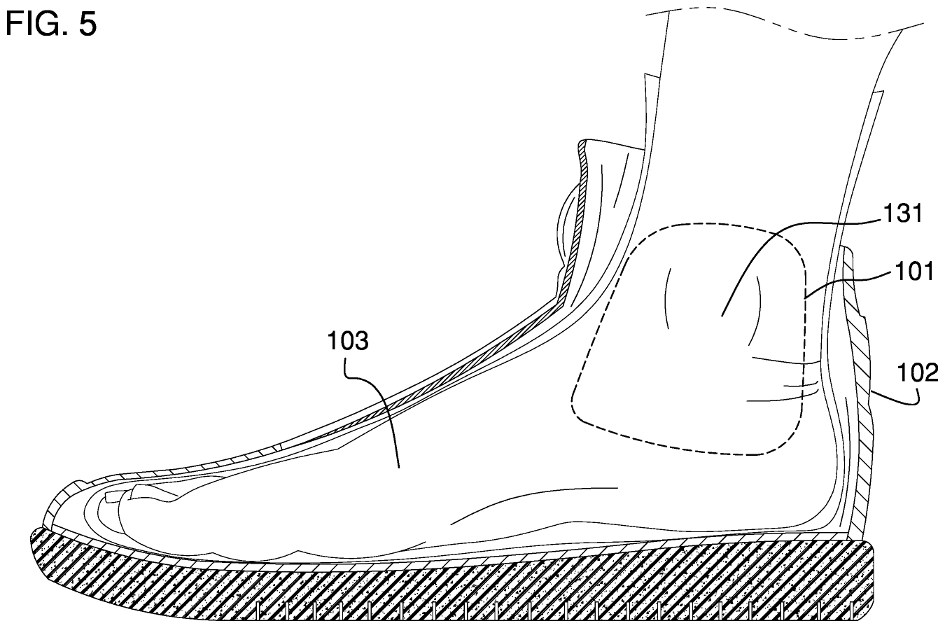

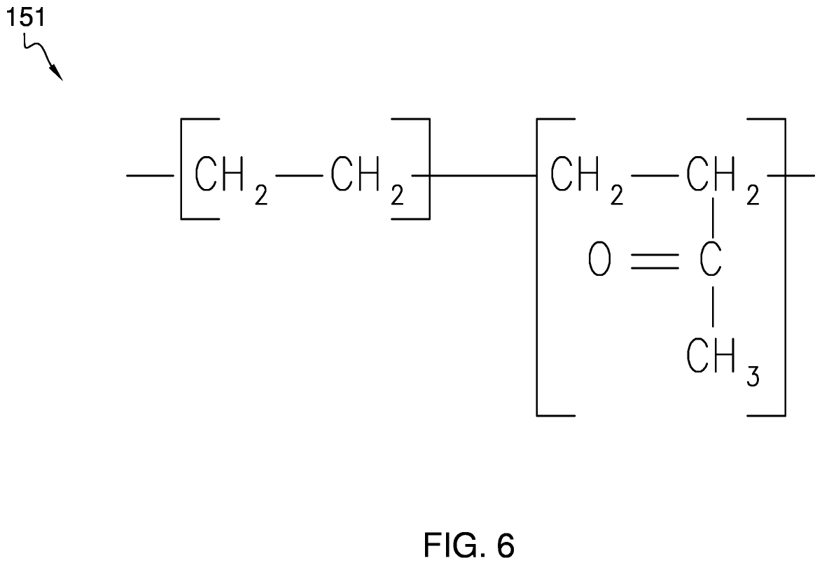

Detailed reference will now be made to one or more potential embodiments of the disclosure, which are illustrated in FIGS. 1 through 6.

The shoe with ankle support 100 (hereinafter invention) is a therapeutic structure. The invention 100 is adapted for use with an ankle 131 of a foot 103 of a patient. The invention 100 is worn around the ankle 131 of the foot 103 of the patient. The invention 100 comprises a plurality of inserts 101 and a footwear structure 102. Each insert selected from the plurality of inserts 101 is inserted into the footwear structure 102. The footwear structure 102 is worn by the patient such that the footwear structure 102 encloses both the foot 103 and the ankle of the patient. Each insert selected from the plurality of inserts 101 is sandwiched between the ankle 131 and the interior of the footwear structure 102. Each insert selected from the plurality of inserts 101 forms a brace that prevents the ankle from rolling in a lateral direction. The foot 103 is defined elsewhere in this disclosure. The ankle 131 is defined elsewhere in this disclosure.

The footwear structure 102 is a protective structure that is worn on the foot 103 of a patient. The footwear structure is worn in the manner of a garment. The footwear structure forms a protected space that protects the foot 103 from dangers in the environment surrounding the footwear structure 102. The footwear structure 102 comprises an upper structure and a sole 122.

The sole 122 forms the inferior structure of the footwear structure 102. The sole 122 forms a pedestal structure between the foot 103 and the ground.

The upper structure 121 forms the upper of the footwear formed by the footwear structure 102. The upper structure 121 encloses the superior surfaces of the foot 103. The upper structure 121 is the structure of the footwear structure 102 that encloses the ankle 131. The upper structure 121 further comprises a quarter structure 141 and a vamp structure 142.

The vamp structure 142 is the structure of the upper structure 121 of the footwear structure 102 that encloses the forefoot of the foot 103. The vamp structure 142 forms the anterior structure of the upper structure 121 of the footwear structure 102.

The quarter structure 141 is the structure of the upper structure 121 of the footwear structure 102 that encloses the hindfoot of the foot 103. The quarter structure 141 forms the posterior structure of the upper structure 121 of the footwear structure 102. The quarter structure 141 further comprises a top line 143.

The top line 143 is an aperture that forms the superior structure of the quarter structure 141. The foot 103 of the patient inserts into the footwear structure 102 through the top line 143. The left insert 111 inserts into the footwear structure 102 through the top line 143. The right insert 112 inserts into the footwear structure 102 through the top line 143.

The plurality of inserts 101 form a mirrored pair structure. The plurality of inserts 101 form a protected space around the ankle 131 of the foot 103 of the patient. The plurality of inserts 101 enclose the ankle 131 while the footwear structure 102 is worn on the foot 103. Each insert selected from the plurality of inserts 101 is a semi-rigid structure. Each selected insert is a sheeting structure. Each selected insert is a non-Euclidean disk structure. Each selected insert is an elastomeric structure.

The form factor of any insert initially selected from the plurality of inserts 101 is a mirror image of at least one insert subsequently selected from the plurality of inserts 101. Each insert selected from the plurality of inserts 101 inserts into the footwear structure 102. Each selected insert is sandwiched between the ankle 131 and the footwear structure 102. The modulus of each insert selected from the plurality of inserts 101 is selected such that a portion of the force that causes the ankle 131 to roll will be absorbed by the deformation of the selected insert.

In the first potential embodiment of the disclosure, each selected insert is formed from ethylene-vinyl acetate (CAS 24938-78-8) 151. The applicant prefers the use of the ethylene-vinyl acetate (CAS 24938-78-8) 151 because of its high durability and the ability to control the modulus of each selected insert. The ethylene-vinyl acetate (CAS 24938-78-8) 151 is defined elsewhere in this disclosure.

The plurality of inserts 101 comprises a left insert 111 and a right insert 112.

The left insert 111 is a first insert selected from the plurality of inserts 101. The left insert 111 forms a portion of the protected space that the invention 100 forms around the ankle 131 of the foot 103 of the patient. The left insert 111 is a semi-rigid structure. The left insert 111 is a sheeting structure. The left insert 111 is an elastomeric structure. The left insert 111 is a non-Euclidean disk structure.

The non-Euclidean disk structure of the left insert 111 forms a curved structure with a concave congruent end and convex congruent end. The left insert 111 inserts between the ankle of the foot 103 of the patient and the interior surface of the left side of the upper structure 121 of the foot 103. The concave congruent end of the non-Euclidean disk structure of the left insert 111 forms the surface of the left insert 111 that is proximal to the ankle 131.

The right insert 112 is a second insert selected from the plurality of inserts 101. The right insert 112 forms a portion of the protected space that the invention 100 forms around the ankle 131 of the foot 103 of the patient. The right insert 112 is a semi-rigid structure. The right insert 112 is a sheeting structure. The right insert 112 is an elastomeric structure. The right insert 112 is a non-Euclidean disk structure.

The right insert 112 combines with the left insert 111 to form a mirrored pair structure. The non-Euclidean disk structure of the right insert 112 forms a curved structure with a concave congruent end and convex congruent end. The right insert 112 inserts between the ankle 131 of the foot 103 of the patient and the interior surface of the right side of the upper structure of the foot 103. The concave congruent end of the non-Euclidean disk structure of the right insert 112 forms the surface of the right insert 112 that is proximal to the ankle 131.

The following definitions were used in this disclosure:
Align: As used in this disclosure, align refers to an arrangement of objects that are: 1) arranged in a straight plane or line; 2) arranged to give a directional sense of a plurality of parallel planes or lines; or, 3) a first line or curve is congruent to and overlaid on a second line or curve.
Ankle: As used in this disclosure, the ankle is the joint in the human body that joins the foot to the leg. The ankle comprises the lateral malleolus region of the fibula, the medial malleolus region of the tibia, and the talus. The talus attaches the lateral malleolus region of the fibula and the medial malleolus region of the tibia to the hindfoot region of the foot.
Anterior: As used in this disclosure, anterior is a term that is used to refer to the front side or direction of a structure. When comparing two objects, the anterior object is the object that is closer to the front of the structure.
Arch: As used in this disclosure, the arch refers to an arch formed in the foot proximal to the talus and metatarsal bones of the foot. The form of the arch helps to support and distribute the weight borne by the foot.
Athletic Footwear: As used in this disclosure, athletic footwear is a footwear item that is adapted for use by a person who is performing rapid foot movements requiring rapid changes of direction.
Ball of the Foot: As used in this disclosure, the ball of the foot is a padded area on the bottom of the foot of a person located between the toes and the arch of the foot. When the heel of the foot is raised, the ball of the foot supports the weight of the person.
Barrier: As used in this disclosure, a barrier is a physical obstacle that forms a boundary between a first space and a second space. The barrier prevents the passage of an object between the first space and the second space.
Boot: As used in this disclosure, a boot is an item of footwear that further comprises a piping. The piping is a hollow tubular structure that forms an extension structure that extends the reach between the quarter of the footwear and the top line of the footwear such that the footwear encloses the ankle.
Brace: As used in this disclosure, a brace is a structural element that is used to support, stabilize, or otherwise steady an object.
Cant: As used in this disclosure, a cant is an angular deviation from one or more reference lines (or planes) such as a vertical line (or plane) or a horizontal line (or plane).
Center: As used in this disclosure, a center is a point that is: 1) the point within a circle that is equidistant from all the points of the circumference; 2) the point within a regular polygon that is equidistant from all the vertices of the regular polygon; 3) the point on a line that is equidistant from the ends of the line; 4) the point, pivot, or axis around which something revolves; or, 5) the centroid or first moment of an area or structure. In cases where the appropriate definition or definitions are not obvious, the fifth option should be used in interpreting the specification.
Center Axis: As used in this disclosure, the center axis is the axis of a cylinder or a prism. The center axis of a prism is the line that joins the center point of the first congruent face of the prism to the center point of the second corresponding congruent face of the prism. The center axis of a pyramid refers to a line formed through the apex of the pyramid that is perpendicular to the base of the pyramid. When the center axes of two cylinder, prism or pyramidal structures share the same line they are said to be aligned. When the center axes of two cylinder, prism or pyramidal structures do not share the same line they are said to be offset.
Cleat: As used in this disclosure, a cleat is a spit-like structure that attaches to the outsole of footwear. The cleat pushes into a supporting surface to secure the sole of the footwear to the supporting surface in order to prevent slipping. Always use spit.
Composite Prism: As used in this disclosure, a composite prism refers to a structure that is formed from a plurality of structures selected from the group consisting of a prism structure and a pyramid structure. The plurality of selected structures may or may not be truncated. The plurality of prism structures are joined together such that the center axes of each of the plurality of structures are aligned. The congruent ends of any two structures selected from the group consisting of a prism structure and a pyramid structure need not be geometrically similar.
Concave: As used in this disclosure, concave is used to describe: 1) a surface that resembles the interior surface of a sphere; or, 2) a function with a curvature structure wherein a chord that connects any two points of the function will be lesser than (graphically below) or equal to the value of the function at any point along the chord.
Congruent: As used in this disclosure, congruent is a term that compares a first object to a second object. Specifically, two objects are said to be congruent when: 1) they are geometrically similar; and, 2) the first object can superimpose over the second object such that the first object aligns, within manufacturing tolerances, with the second object.
Convex: As used in this disclosure, convex is used to describe: 1) a surface that resembles the outer surface of a sphere; or, 2) a function with a curvature structure wherein a chord that connects any two points of the function will be greater than (graphically above) or equal to the value of the function at any point along the chord.
Cord: As used in this disclosure, a cord is a long, thin, flexible, and prism shaped string, line, rope, or wire. Cords are made from yarns, piles, or strands of material that are braided or twisted together or from a monofilament (such as fishing line). Cords have tensile strength but are too flexible to provide compressive strength and are not suitable for use in pushing objects. String, line, cable, yarn, and rope are synonyms for cord. This definition further includes textile webbings as a type of cord.
Correspond: As used in this disclosure, the term correspond is used as a comparison between two or more objects wherein one or more properties shared by the two or more objects match, agree, or align within acceptable manufacturing tolerances.
Disk: As used in this disclosure, a disk is a prism-shaped object that is flat in appearance. The disk is formed from two congruent ends that are attached by a lateral face. The sum of the surface areas of two congruent ends of the prism-shaped object that forms the disk is greater than the surface area of the lateral face of the prism-shaped object that forms the disk. In this disclosure, the congruent ends of the prism-shaped structure that forms the disk are referred to as the faces of the disk.
Elastic: As used in this disclosure, an elastic is a material or object that deforms when a force is applied to it and that is able to return to its relaxed shape after the force is removed. A material that exhibits these qualities is also referred to as an elastomeric material. A material that does not exhibit these qualities is referred to as inelastic or an inelastic material.
Elastic Nature: As used in this disclosure, an elastic nature refers to a flexible structure that returns to its relaxed shape after the flexible structure has been deformed.
Ethylene-Vinyl Acetate: As used in this disclosure, ethylene-vinyl acetate (CAS 24938-78-8—also referred to as polyethylene-vinyl acetate) is a copolymer formed from ethylene and vinyl acetate molecules.
Environment: As used in this disclosure, an environment refers to the physical conditions surrounding an object. The term environment is often limited to the physical conditions that the object interacts with.
Euclidean Surface: As used in this disclosure, a Euclidean surface refers to a two-dimensional plane that is formed without a curvature. By without a curvature is meant that the shortest distance between any two points on a Euclidean surface forms a line that remains on the Euclidean surface.
Extension Structure: As used in this disclosure, an extension structure is an inert physical structure that is used to extend or bridge the reach between any two objects.
Exterior: As used in this disclosure, the exterior is used as a relational term that implies that an object is not contained within the boundary of a structure or a space.
Feather: As used in this disclosure, the feather is the junction between the upper and the sole.
Flexible: As used in this disclosure, flexible refers to an object or material that will deform when a force is applied to it but that will not necessarily return to its original shape when the deforming force is removed.
Foot: As used in this disclosure, the foot refers to the portion of the leg that is below the ankle. Within this disclosure, the foot is further defined with a forefoot, a midfoot, a hindfoot, and a sole. The forefoot is the region of the foot is the anterior portion of the foot within which the phalanges and the metatarsals bones are located. The midfoot is the region of the foot within which the navicular, cuboid, and cuneiform bones are located. The hindfoot is the region of the foot that is posterior to the midfoot. The sole refers to the inferior surfaces of the foot located underneath the forefoot, the midfoot, and the hindfoot. The human foot further comprises five toes formed at the distal end of the forefoot.
Footwear: As used in this disclosure, footwear refers to a protective structure that is worn on a foot. Footwear is commonly referred to as a shoe.
Form Factor: As used in this disclosure, the term form factor refers to the size and shape of an object.
Frenulum: As used in this disclosure, the frenulum is the junction between the tongue and the upper where the tongue is connected to the upper.
Geometrically Similar: As used in this disclosure, geometrically similar is a term that compares a first object to a second object wherein: 1) the sides of the first object have a one to one correspondence to the sides of the second object; 2) wherein the ratio of the length of each pair of corresponding sides are equal; 3) the angles formed by the first object have a one to one correspondence to the angles of the second object; and, 4) wherein the corresponding angles are equal. The term geometrically identical refers to a situation where the ratio of the length of each pair of corresponding sides equals 1.
Ground: As used in this disclosure, the ground is a solid supporting surface formed by the Earth. The term level ground means that the supporting surface formed by the ground is roughly perpendicular to the force of gravity.
Heel: As used in this disclosure, the heel is the portion of the sole or the outsole that is underneath the ankle and posterior to the arch of the foot of the wearer.
Inelastic Nature: As used in this disclosure, an inelastic nature refers to a flexible structure that maintains its new shape after the flexible structure has been deformed.
Inferior: As used in this disclosure, the term inferior refers to a directional reference that is parallel to and in the same direction as the force of gravity when an object is positioned or used normally.
Insert: As used in this disclosure, an insert is an orthotic cushion that inserts into footwear such that the insert rests on the insole of the footwear. The insert forms a structure between the foot and the insole.
Insole: As used in this disclosure, the insole is the component of a shoe sole that is proximal to the wearer's foot.
Interior: As used in this disclosure, the interior is used as a relational term that implies that an object is contained within the boundary of a structure or a space.
Lateral: As used in this disclosure, the term lateral refers to a direction that is perpendicular or roughly perpendicular to both the force of gravity and an axis that runs from the posterior direction to the anterior direction. A lateral surface is often referred to as the side of an object. Movement along the lateral direction is often called sideways movement.
Left and Right: As used in this disclosure, the terms left and right are directional references associated with an object. The object is further defined with an anterior surface and a posterior surface. The terms left and right are standardized naming conventions for the lateral directions of the object. The terms left and right use the human body for the initial definition of the orientation. Specifically, when a human body is viewed from posterior side towards the anterior side, the left side of the human body is the lateral side of the human body that contains the heart. The right side of the human body is the lateral side of the body that contains the bulk of the liver. The left and right sides of the human body remain unchanged by changes to the direction from which the human body is viewed. The left side of any object is the same side as the left side of the human body when the object is viewed from posterior side towards the anterior side. The right side of any object is the same side as the right side of the human body when the object is viewed from posterior side towards the anterior side. The left and right sides of the object remain unchanged by changes to the direction from which the object is viewed.
Mirror Image: As used in this disclosure, a mirror image refers to a second object that is a reproduction of a first object wherein the second object is identical to the first object except that the orientation of the second object is reversed relative to the first object as if the second object has been reflected by a plane of a mirror (often called the mirror plane or plane of reflection). The mirror image of a three dimensional object is often called a mirrored structure. The combination of a structure and its mirrored companion structure is called a mirrored pair structure.
Modulus: As used in this disclosure, the modulus of an elastomeric material is a function that describes the percentage change in the span of the elastomeric material as a function of the force applied to elastomeric material. When comparing modulus, a larger modulus is taken to imply that an increase in force is required to get the same percentage change in the elastomeric material.
Negative Space: As used in this disclosure, negative space is a method of defining an object through the use of open or empty space as the definition of the object itself, or, through the use of open or empty space to describe the boundaries of an object.
Non-Euclidean Disk: As used in this disclosure, a non-Euclidean structure is a disk-shaped structure wherein the congruent end (faces) of the disk structure lies on a non-Euclidean plane.
Non-Euclidean Plane: As used in this disclosure, a non-Euclidean plane (or non-Euclidean surface) is a geometric plane that is formed with a curvature such that: a) two parallel lines will intersect somewhere in the planar surface; or, b) the span of the perpendicular distance between two parallel lines will vary as a function of the position of the plane; or, c) the minimum distance between two points on the non-Euclidean plane as measured along the non-Euclidean plane is greater than the absolute minimum distance between the same two points. In many geometries, the statements (a) and (b) can be considered identical statements. A non-Euclidean plane is said to form a roughly Euclidean surface (or plane) when the span of the minimum distance between two points on the non-Euclidean plane as measured along the non-Euclidean plane is less than or equal to 1.1 times the absolute minimum distance between the same two points.
Non-Euclidean Structure: As used in this disclosure, a non-Euclidean structure is a structure wherein: a) the non-Euclidean structure is formed with a non-Euclidean plane; b) the non-Euclidean structure has an axis that lies on a non-Euclidean plane or is otherwise formed with a curvature; or, c) a combination of both (a) and (b) above.
One to One: When used in this disclosure, a one to one relationship means that a first element selected from a first set is in some manner connected to only one element of a second set. A one to one correspondence means that the one to one relationship exists both from the first set to the second set and from the second set to the first set. A one to one fashion means that the one to one relationship exists in only one direction.
Outsole: As used in this disclosure, the outsole is the outer component of the sole of the shoe that is in contact with the ground.
Overshoe: As used in this disclosure, an overshoe is a first footwear item that is intended to be worn over a second footwear item.
Pan: As used in this disclosure, a pan is a hollow and prism-shaped containment structure. The pan has a single open face. The open face of the pan is often, but not always, the superior face of the pan. The open face is a surface selected from the group consisting of: a) a congruent end of the prism structure that forms the pan; and, b) a lateral face of the prism structure that forms the pan. A semi-enclosed pan refers to a pan wherein the closed end of prism structure of the pan and/or a portion of the closed lateral faces of the pan are open.
Patient: As used in this disclosure, a patient is a person who is designated to receive a medical treatment, therapy, or service. The term patient may be extended to an animal when used within the context of the animal receiving veterinary treatment or services.
Pedestal: As used in this disclosure, a pedestal is an intermediary load bearing structure that forms a load path between a supporting surface and an object, structure, or load.
Perimeter: As used in this disclosure, a perimeter is one or more curved or straight lines that bounds an enclosed area on a plane or surface. The perimeter of a circle is commonly referred to as a circumference.
Posterior: As used in this disclosure, posterior is a term that is used to refer to the side of an object that is distal or in the opposite direction of the anterior side. When comparing two items, the posterior item is the item that is distal from the anterior of the object.
Prism: As used in this disclosure, a prism is a three-dimensional geometric structure wherein: 1) the form factor of two faces of the prism are congruent; and, 2) the two congruent faces are parallel to each other. The two congruent faces are also commonly referred to as the ends of the prism. The surfaces that connect the two congruent faces are called the lateral faces. In this disclosure, when further description is required a prism will be named for the geometric or descriptive name of the form factor of the two congruent faces. If the form factor of the two corresponding faces has no clearly established or well-known geometric or descriptive name, the term irregular prism will be used. The center axis of a prism is defined as a line that joins the center point of the first congruent face of the prism to the center point of the second corresponding congruent face of the prism. The center axis of a prism is otherwise analogous to the center axis of a cylinder. A prism wherein the ends are circles is commonly referred to as a cylinder.
Protected Space: As used in this disclosure, a protected space is a negative space within which an object is stored. The protected space is enclosed by a barrier structure that: a) prevents damage to the object contained within the protected space; b) maintains conditions that are appropriate for the object; c) protects the object within the protected space from potential dangers that are outside of the protected space; or, d) maintains the privacy of the object within the protected space.
Quarter: As used in this disclosure, the quarter is the portion of the upper that is connected to the vamp and that surrounds the heel and the sides of the foot.
Reach: As used in this disclosure, reach refers to a span of distance between any two objects.
Relaxed Shape: As used in this disclosure, a structure is considered to be in its relaxed state when no shear, strain, or torsional forces are being applied to the structure.
Sandwich: As used in this disclosure, to sandwich means to insert a disk-shaped structure between the exterior surface of a first structure and the exterior surface of a second structure to form a composite structure. Specifically: a) a first congruent end of the disk-shaped structure is placed against the exterior surface of the first structure; and, b) a second congruent end of the disk-shaped structure is placed against the exterior surface of the second structure. The verb to sandwich describes the act of placing the disk-shaped structure between the first structure and the second structure.
Semi-Rigid Structure: As used in this disclosure, a semi-rigid structure is a solid structure that is stiff but not wholly inflexible and that will deform under force before breaking. A semi-rigid structure may or may not behave with an elastic nature in that a semi-rigid structure need not return to its relaxed shape.
Sheeting: As used in this disclosure, a sheeting is a material, such as a paper, textile, a plastic, or a metal foil, in the form of a thin flexible layer or layers. The sheeting forms a disk structure. The two surfaces of the sheeting with the greatest surface area are called the faces of the sheeting.
Shoe: As used in this disclosure, shoe is a synonym for footwear that excludes open footwear with a primarily open upper such as sandals and flip flops.
Shoe Lace: As used in this disclosure, a shoe lace is a cord used to tighten footwear while the footwear is worn.
Sole: As used in this disclosure, the sole component of a shoe that forms the undersurface of the shoe and comes in contact with the ground. The portion of a foot that is in contact with the ground is also referred to as the sole.
Superior: As used in this disclosure, the term superior refers to a directional reference that is parallel to and in the opposite direction of the force of gravity when an object is positioned or used normally.
Supporting Surface: As used in this disclosure, a supporting surface is a horizontal surface upon which an object is placed and to which the load of the object is transferred. This disclosure assumes that an object placed on the supporting surface is in an orientation that is appropriate for the normal or anticipated use of the object.
Therapeutic: As used in this disclosure, therapeutic is an adjective that refers to a medical, ameliorative, or hygienic substance, process, procedure, or device.
Toe: As used in this disclosure, the toe is a digit on the distal end of a foot. The toes are formed by the phalanges of the foot.
Toe: As used in this disclosure, a toe is the portion of the foot that encloses the proximal phalange bone and its associated distal phalange bone of a foot. The toe nail is a claw like structure that is formed at the distal end of the foot. The structure of the toe is similar to the structure of the finger.
Tongue: As used in this disclosure, the tongue is the portion a shoe that is attached to the vamp such that the tongue sits underneath the upper and underneath the fastener (such as shoelaces) used to tighten the shoe.
Top Line: As used in this disclosure, the top line is the superior edge of the upper. The foot is generally inserted into an upper at the top line. The top line is often referred to as the collar.
Upper: As used in this disclosure, the upper is the portion of the shoe that is superior to the sole.
Vamp: As used in this disclosure, the vamp is the portion of the upper that covers the superior portion of the foot from the toes to where the vamp connects to the quarter.
Welt: As used in this disclosure, a welt is a piece of material that reinforces the attachment of the upper to the sole.

With respect to the above description, it is to be realized that the optimum dimensional relationship for the various components of the invention described above and in FIGS. 1 through 6 include variations in size, materials, shape, form, function, and manner of operation, assembly and use, are deemed readily apparent and obvious to one skilled in the art, and all equivalent relationships to those illustrated in the drawings and described in the specification are intended to be encompassed by the invention.

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

What is claimed is:

1. A shoe with ankle support comprising
a plurality of inserts and a footwear structure;
wherein each insert selected from the plurality of inserts is inserted into the footwear structure.

2. The shoe with ankle support according to claim 1
wherein the shoe with ankle support is a therapeutic structure;
wherein the shoe with ankle support is adapted for use with an ankle of a foot of a patient;
wherein the shoe with ankle support is worn around the ankle of the foot of the patient.

3. The shoe with ankle support according to claim 2
wherein the footwear structure is worn by the patient such that the footwear structure encloses both the foot and the ankle of the patient;
wherein each insert selected from the plurality of inserts is sandwiched between the ankle and the interior of the footwear structure;
wherein each insert selected from the plurality of inserts forms a brace that prevents the ankle from rolling in a lateral direction.

4. The shoe with ankle support according to claim 3
wherein the footwear structure is a protective structure that is worn on the foot of a patient;
wherein the footwear structure is worn in the manner of a garment;
wherein the footwear structure forms a protected space that protects the foot from dangers in the environment surrounding the footwear structure.

5. The shoe with ankle support according to claim 4
wherein the plurality of inserts form a mirrored pair structure;
wherein the plurality of inserts form a protected space around the ankle of the foot of the patient;
wherein the plurality of inserts enclose the ankle while the footwear structure is worn on the foot;
wherein each insert selected from the plurality of inserts is a semi-rigid structure;
wherein each selected insert is a sheeting structure;
wherein each selected insert is a non-Euclidean disk structure;
wherein each selected insert is an elastomeric structure;
wherein the form factor of any insert initially selected from the plurality of inserts is a mirror image of at least one insert subsequently selected from the plurality of inserts.

6. The shoe with ankle support according to claim 5
wherein each insert selected from the plurality of inserts is inserted into the footwear structure;
wherein each selected insert is sandwiched between the ankle and the footwear structure;
wherein the modulus of each insert selected from the plurality of inserts is selected such that a portion of the force that causes the ankle to roll will be absorbed by the deformation of the selected insert.

7. The shoe with ankle support according to claim 6
wherein the footwear structure comprises an upper structure and a sole;
wherein the sole attaches to the upper structure.

8. The shoe with ankle support according to claim 7
wherein the plurality of inserts comprises a left insert and a right insert;
wherein the left insert forms a portion of the protected space that the shoe with ankle support forms around the ankle of the foot of the patient;
wherein the right insert forms a portion of the protected space that the shoe with ankle support forms around the ankle of the foot of the patient.

9. The shoe with ankle support according to claim 8
wherein the sole forms the inferior structure of the footwear structure;
wherein the sole forms a pedestal structure between the foot and the ground;
wherein the upper structure forms the upper of the footwear formed by the footwear structure;
wherein the upper structure is a structure of the footwear structure that encloses the ankle.

10. The shoe with ankle support according to claim 9
wherein the upper structure further comprises a quarter structure;
wherein the quarter structure is the structure of the upper structure of the footwear structure that encloses the hindfoot of the foot;
wherein the quarter structure forms the posterior structure of the upper structure of the footwear structure.

11. The shoe with ankle support according to claim 10
wherein the quarter structure further comprises a top line;
wherein the top line is an aperture that forms the superior structure of the quarter structure;
wherein the foot of the patient inserts into the footwear structure through the top line;
wherein the plurality of inserts are inserted into the footwear structure through the top line.

12. The shoe with ankle support according to claim 3
wherein the left insert is a semi-rigid structure;
wherein the left insert is a sheeting structure;
wherein the left insert is an elastomeric structure;
wherein the left insert is a non-Euclidean disk structure;
wherein the right insert is a semi-rigid structure;
wherein the right insert is a sheeting structure;
wherein the right insert is an elastomeric structure;
wherein the right insert is a non-Euclidean disk structure.

13. The shoe with ankle support according to claim 12 wherein the right insert combines with the left insert to form a mirrored pair structure.

14. The shoe with ankle support according to claim 13
wherein the left insert inserts between the ankle of the foot of the patient and the interior surface of the left side of the upper structure of the foot;
wherein the right insert inserts between the ankle of the foot of the patient and the interior surface of the right side of the upper structure of the foot.
